(19) United States
(12) Patent Application Publication
(10) Pub. No.: US 2023/0070032 A1
(43) Pub. Date: Mar. 9, 2023

(54) SOLID CANNABIDIOL COMPOSITIONS

(71) Applicant: Feals, Inc., Denver, CO (US)

(72) Inventor: Drew Todd Puchalski, Los Angeles, CA (US)

(73) Assignee: Feals, Inc., Denver, CO (US)

(21) Appl. No.: 17/894,830

(22) Filed: Aug. 24, 2022

Related U.S. Application Data
(60) Provisional application No. 63/236,472, filed on Aug. 24, 2021.

(51) Int. Cl. A61K 31/05; A61K 47/14; A61K 36/068; A61K 47/36; A61K 9/20; A61K 36/185

(52) U.S. Cl. CPC A61K 31/05; A61K 47/14; A61K 36/068; A61K 47/36; A61K 9/2095; A61K 36/185

(57) ABSTRACT

The present invention discloses orally dissolvable solid CBD compositions comprising encapsulated CBD, mushroom mycelia, and flavor.

### CROSS-REFERENCE TO PRIOR APPLICATION

This application claims priority benefit from U.S. Provisional Patent Application No. 63/236,472, filed on Aug. 24, 2021, the entire content of which is incorporated herein by reference. All references cited anywhere in this specification, including the Background and Detailed Description sections, are incorporated by reference as if each had been individually incorporated.

### FIELD OF INVENTION

The present invention is directed to an orally dissolvable solid CBD composition.

### SUMMARY

One general aspect of the invention is a solid composition that includes encapsulated cannabidiol (CBD), mushroom mycelia, and flavor. The solid composition can contain about 20 wt. % of the encapsulated CBD, and the encapsulated CBD can contain about 10 wt. % of CBD. The CBD can be selected from CBD distillate, CBD isolate, full-spectrum CBD oil, and broad-spectrum CBD oil. The encapsulated CBD includes a CBD emulsion that is spray dried on or in a substrate. The substrate can be maltodextrin, such as digestion resistant maltodextrin (DRM). The mushroom mycelia can be selected from Cordyceps sinensis mycelia. The flavor may include a sweetener and an optional flavorant. The sweetener can be stevia. Solid composition can be free of sucrose. The solid composition can be a tablet. The solid composition can be orally dissolvable.

Another general aspect of the invention is a method of masking the bitter taste of CBD. The method of masking includes i) preparing a CBD emulsion that includes a CBD and a carrier oil; ii) spray drying the emulsion onto a substrate to obtain a powder; and iii) combining the powder with mushroom mycelia. The CBD can be selected from CBD distillate, CBD isolate, full-spectrum CBD oil, and broad-spectrum CBD oil. The substrate can be maltodextrin, such as digestion resistant maltodextrin (DRM). The mushroom mycelia can be selected from Cordyceps sinensis mycelia. The carrier oil can be medium chain triglyceride (MCT) oil.

Another general aspect of the invention is a nutraceutical product that may include the solid composition. The nutraceutical product can contain about 20 wt. % of the encapsulated CBD, and the encapsulated CBD can contain about 10 wt. % of CBD. The nutraceutical product can be a mint.

### DETAILED DESCRIPTION

Embodiments of the invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. All references cited herein are incorporated by reference as if each had been individually incorporated.

Unless otherwise indicated, all parts and percentages are by weight. As used herein, the term “about” refers to plus or minus 10% of the indicated value. Unless otherwise stated or made clear by context, weight percentages are provided based on the total amount of the composition in which they are described. As used herein, the singular forms “a,” “an,” and “the” include plural referents unless the context clearly dictates otherwise.

Described herein are solid CBD compositions containing cannabidiol (CBD), mushroom mycelia, and flavor, together with additional formulation excipients.

Cannabidiol (CBD)

The solid CBD compositions of the present invention include cannabidiol, also known as CBD. CBD is one of about 113 identified cannabinoids identified in cannabis plants, and accounts for up to 40% of the plant's extract. Clinical research has indicated CBD has therapeutic implications in treatment of anxiety, cognition, movement disorders, and pain.

Any source of CBD can be used in the solid CBD compositions of the present invention. In embodiments of the invention, the CBD is selected from CBD isolate, CBD distillate, broad-spectrum CBD oil, or full-spectrum CBD oil. CBD isolate contains only CBD. CBD distillate, broad-spectrum CBD oil, and full-spectrum CBD oil predominantly contain CBD, but also include a variety of other organic compounds found in the cannabis plant that help generate the “entourage effect.” In particular, broad-spectrum CBD oil contains up to 0.3% delta-9 THC (tetrahydrocannabinol) whereas full spectrum CBD oil does not contain delta-9 THC.

The “entourage effect” occurs when CBD is ingested with other compounds, such as other cannabinoids and terpenes, from the cannabis plant. By using compounds from the whole hemp plant, such as in full-spectrum or broad-spectrum CBD oil, rather than a singular aspect or compound, there is a boost in the health benefits and overall therapeutic effect of cannabinoids. This results in a higher likelihood of pain relief, anti-inflammatory action, and anxiety relief.

The solid CBD compositions of the present invention can include encapsulated CBD. Any type of encapsulated CBD can be used in the present invention, including encapsulated CBD isolate, encapsulated CBD distillate, encapsulated broad-spectrum CBD oil, or encapsulated full-spectrum CBD oil.

Encapsulated CBD is typically made by creating a CBD nanoemulsion which is then spray dried onto a substrate. In embodiments of the invention, the nanoemulsion is prepared without an emulsifier or emulsifying agent by using microfluidizer technology. In this embodiment, the nanoemulsion is prepared by combining an oil phase containing the CBD source, for example full-spectrum or broad-spectrum CBD oil, with an aqueous phase of deionized (DI) water. In exemplary embodiments, the CBD source or CBD oil is dissolved in a carrier oil before mixing with water. An exemplary carrier oil is MCT (Medium Chain Triglycerides) oil, although other oils may be used are acceptable. Both phases are heated to assist and maintain the dissolution of the CBD source in the carrier oil.

A coarse emulsion is made by slowly introducing the oil phase to the aqueous phase and mixing with a low shear rotor-stator mixed to create a coarse or pre-emulsion. The coarse emulsion is stable enough to not experience phase separation prior to processing in the microfluidizer processor. The coarse emulsion is then passed through the microfluidizer processor a number of times and converted into a nanoemulsion.

Once the emulsion process is complete, the encapsulated CBD is formed by spraying drying the CBD nanoemulsion onto a substrate. In an alternative embodiment, the substrate is mixed with the CBD nanoemulsion and the combination spray dried together. In embodiments of the invention, the substrate is a maltodextrin. In exemplary embodiments of the invention, the maltodextrin is a digestion resistant maltodextrin (DRM). DRM is over 90% fiber and used for fiber enrichment in a variety of foods as a soluble fiber. It is used to replace the functionality of sugar, and used for sugar and calorie reduction. It is soluble in water and made from corn starch. An exemplary DRM is Fibersol 2, which available from Fibersol®.

In embodiments of the invention, the CBD nanoemulsion is prepared without an emulsifier, and the encapsulated CBD does not contain an emulsifier. In particular embodiments of the invention, the encapsulated CBD does not contain purity gum, gum arabic, or modified gum arabic as emulsifiers or taste masking agents.

In embodiments of the invention, the solid CBD compositions of the present invention can contain about 5 wt. % to about 30 wt. % of encapsulated CBD. For example, the solid CBD compositions can contain about 5 wt. %, about 10 wt. %, about 15 wt. %, about 20 wt. %, about 25 wt. %, or about 30 wt. % of encapsulated CBD. In a preferred embodiment, the solid CBD compositions contain about 15 wt. % to about 20 wt. % encapsulated CBD. For example, about 15 wt. % encapsulated CBD.

The encapsulated CBD contains about 10-20% of CBD isolate, CBD distillate, broad-spectrum CBD oil, or full-spectrum CBD oil. The encapsulated CBD has no oily surfactants, and no bitter taste. In a preferred embodiment the encapsulated CBD contains about 10% CBD.

Therefore, the solid CBD compositions of the present invention contain about 0.5 wt. % to about 6.0 wt. % of CBD. For example, the compositions can contain about 0.5 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, or about 6.0 wt. % CBD. In a preferred embodiment, the solid CBD compositions contain about 1.5 wt. % to about 2.0 wt. % CBD. For example, about 1.5 wt. % CBD.

Mushroom Mycelia

The solid CBD compositions of the present invention include mushroom mycelia. The mushroom mycelia function as a bitter blocker by temporarily occupying taste receptors on the tongue so that bitter flavors associated with CBD are unable to bind with the taste receptors. Without an active site to bind, the negative flavors are not detected.

In embodiments of the invention, the mushroom mycelia are Cordyceps sinensis mycelia. An exemplary source of Cordyceps sinensis mycelia is ClearIQBitter Block from MycoTechnology.

In embodiments of the invention, the solid CBD compositions of the present invention can contain about 0.10 wt. % to about 1.0 wt. % of mushroom mycelia. For example, the solid CBD compositions can contain about 0.10 wt. %, about 0.20 wt. %, about 0.30 wt. %, about 0.40 wt. %, about 0.50 wt. %, about 0.60 wt. %, about 0.70 wt. %, about 0.80 wt. %, about 0.90 wt. %, or about 1.00 wt. % of mushroom mycelia. In a preferred embodiment, the solid CBD compositions contain about 0.50 wt. % mushroom mycelia.

Flavor

The solid CBD compositions of the present invention include flavor. The flavor contains a sweetener and an optional flavorant.

In embodiments of the invention, the sweetener is selected from sugars, such as sucrose, fructose, and glucose, or sugar substitutes, such as monk fruit extract, aspartame, sucralose, saccharin, xylitol, acesulfame k, and stevia. In embodiments of the invention, the solid CBD compositions are preferably sugar free, i.e. contain no added sucrose, fructose, or glucose. In a preferred embodiment, the sweetener is stevia.

In embodiments of the invention, the solid CBD compositions of the present invention can contain about 0.10 wt. % to about 1.0 wt. % of sweetener. For example, the solid CBD compositions can contain about 0.10 wt. %, about 0.20 wt. %, about 0.30 wt. %, about 0.40 wt. %, about 0.50 wt. %, about 0.60 wt. %, about 0.70 wt. %, about 0.80 wt. %, about 0.90 wt. %, or about 1.00 wt. % of sweetener. In a preferred embodiment, the solid CBD compositions contain about 0.40 wt. % sweetener. In a most preferred embodiment, the solid CBD compositions contain about 0.40 wt. % stevia.

In embodiments of the invention, the flavorant is selected from one or more of peppermint, spearmint, wintergreen, vanilla, citrus, cinnamon, ginger, and a cooling enhancer.

In embodiments of the invention, the solid CBD compositions of the present invention can contain about 0.50 wt. % to about 1.50 wt. % of flavorant. For example, the solid CBD compositions can contain about 0.50 wt. %, about 0.60 wt. %, about 0.70 wt. %, about 0.80 wt. %, about 0.90 wt. %, about 1.00 wt. %, about 1.10 wt. %, about 1.20 wt. %, about 1.30 wt. %, about 1.40 wt. %, or about 1.50 wt. % flavorant. In a preferred embodiment, the solid CBD compositions contain about 0.85 wt. % flavorant.

Excipients

The solid CBD compositions of the present invention include additional excipients commonly used in the art, such as tableting compounds and lubricants.

In embodiments of the invention, the solid CBD compositions of the present invention can contain about 75.0 wt. % to about 85.0 wt. % of a tableting compound. For example, the solid CBD compositions can contain about 75.0 wt. %, about 76.0 wt. %, about 77.0 wt. %, about 78.0 wt. %, about 79.0 wt. %, about 80.0 wt. %, about 81.0 wt. %, about 82.0 wt. %, about 83.0 wt. %, about 84.0 wt. %, or about 85.0 wt. % of a tableting compound. In a prefer embodiment, the solid CBD compositions contain about 79.0 wt. % of a tableting compound. In exemplary embodiments, the tableting compound is sorbitol. In other exemplary embodiments, the tableting compound is a combination of sorbitol and xylitol. The ratio of sorbitol to xylitol can vary from about 2:1 to about 1:2, for example from about 2:1 to about 1:1. In embodiments, the ratio of sorbitol to xylitol is 1:1 or 2:1.

In embodiments of the invention, the solid CBD compositions of the present invention can contain about 0.50 wt. % to about 1.50 wt. % of lubricant. For example, the solid CBD compositions can contain about 0.50 wt. %, about 0.60 wt. %, about 0.70 wt. %, about 0.80 wt. %, about 0.90 wt. %, about 1.00 wt. %, about 1.10 wt. %, about 1.20 wt. %, about 1.30 wt. %, about 1.40 wt. %, or about 1.50 wt. % of lubricant. In a preferred embodiment, the solid CBD compositions contain about 0.75 wt. % lubricant. In an exemplary embodiment, the lubricant is magnesium stearate.

Solid CBD Compositions

The solid CBD compositions of the present invention are formulated into oral dosage forms, including tablets. The tablet dosage form has a hardness of 8-20 kilograms. In exemplary embodiments, the tablets are orally dissolvable. Tablets of the present invention can be nutraceuticals for delivering CBD. In exemplary embodiments, the solid CBD composition is a mint. In exemplary embodiments, mints of the present invention are 1 gram tablets containing about 15 to about 20 mg, for example about 15 mg of CBD, and are orally dissolvable.

CBD generally imparts a bitter or unpleasant taste to oral delivery systems. Most available CBD delivery vehicles ingested through the mouth, such as beverages and “edibles,” (for example gummies), are quickly swallowed and intended to have very short oral resident times, i.e., remain only briefly in the oral cavity (mouth). Various technologies, for example the use of a variety of encapsulation techniques and taste modifiers have been used to reduce the bitter taste of CBD. Because these prior delivery systems have a relatively short oral resident time, these strategies are sufficient to make the delivery system satiable by masking the bitter taste of the CBD. However, these technologies do not adequately block the bitter taste or flavor of CBD for products intended to have longer oral resident times.

Encapsulated CBDs taste better than their CBD oil counterparts, and are suitable for beverages. Despite their improved taste, encapsulated CBDs alone are unsuitable for products having a longer oral resident time, particularly with high CBD dosages of about 5 mg to about 30 mg. The longer the encapsulated CBD resides in the mouth, the longer the encapsulated CBD is exposed to digestive enzymes in the saliva. As these digestive enzymes break down the encapsulated CBD, the CBD is free to contact taste buds on the tongue and oral epithelium, making the displeasing, bitter taste of the CBD molecule unavoidable to the consumer.

Bitter tastes and flavors can also sometimes be alleviated with added sugar and flavors. However, consumers are now actively avoiding added sugars in most products in efforts to live healthier lives. Therefore, using added sweeteners and flavors alone to block and mask the bitter flavor of CBD is unappealing. Further, an excessive amount of sweetener would be necessary to combat the bitterness associated the high amount of CBD present in the solid CBD compositions of the present invention. The novel combination of the mushroom mycelia and encapsulated CBD work in concert to mask the bitterness of CBD with a minimal amount of sweeteners and flavors. Similarly, mushroom mycelia alone are unsuitable to block CBD bitterness as an excessive amount of mycelia would be required to mask the bitterness of high dose CBD oral delivery system with higher oral resident times.

In embodiments of the invention, the solid CBD compositions are orally dissolvable. In contrast to other oral delivery systems, the solid CBD compositions of the present invent can have longer oral resident times allowing delivery the CBD through the oral mucosa and directly into the bloodstream. The solid CBD compositions of the present invention are thus able to bypass first-pass metabolism by delivering CBD through the oral mucosa of the mouth. This results in rapid onset of the therapeutic effects of the solid CBD compositions, with therapeutic effects usually felt within 20 minutes of administration of the solid CBD composition. In embodiments of the invention, the therapeutic effects are felt within 5 minutes, within 10 minutes, within 15 minutes, within 20 minutes, within 25 minutes, or within 30 minutes of administration of the solid composition.

In order to successfully deliver the CBD through the oral mucosa of the mouth, the solid CBD compositions contain a water-soluble CBD component. However, since the water-soluble CBD component is prone to contact taste receptors on the tongue and elsewhere in the oral epithelium, the bitter taste of the CBD molecule needs to be masked to make the solid CBD compositions palatable. The solid CBD compositions of the present invention utilize a novel combination of encapsulated CBD together with a mushroom mycelia bitter blocker to simultaneously address the CBD solubility and bitter taste issues.

The solid CBD compositions contain about 5-30 mg of CBD. In order to ensure the majority of the CBD is delivered through the oral mucosa, the solid CBD composition must be held in the mouth for an extended amount of time. The novel combination of the encapsulated CBD with the mushroom mycelia effectively blocks the bitter taste of the CBD from being detected the entire time the solid CBD composition is held in the mouth. This ensures successful delivery of all 5-30 mg of CBD through the oral mucosa.

Table 1 below discloses a general formulation blend for solid CBD compositions according to the invention.

TABLE 1
General formulation

| Component | Range (wt. %) |
|---|---|
| Base | 75.0-85.0 |
| Lubricant | 0.5-1.5 |
| Sweetener | 0.1-1.0 |
| Flavorant | 0.5-1.5 |
| Bitter blocker | 0.1-1.0 |
| Encapsulated CBD | 5.0-30.0 |
| (10% on maltodextrin) | |

Table 2 below discloses an exemplary formulation blend for an orally dissolvable mint according to the invention.

TABLE 2
Exemplary Mint Formulation Blend

| Component | Ingredient | Amount (wt. %) |
|---|---|---|
| Base | Sorbitol tableting blend | 79.08 |
| Lubricant | Magnesium stearate | 0.75 |
| Sweetener | Stevia | 0.4 |
| Flavorant | Peppermint | 0.75 |
| Flavorant | Fona Optify cooling enhancer | 0.1 |
| Bitter blocker | ClearIQ Bitter Blocker | 0.5 |
| Encapsulated CBD | Encapsulated CBD | 18.42 |
| | (10%) on fibersol | |

| | |
|---|---|
| Total | 100.00 |

Table 3 below discloses a second exemplary formulation blend for an orally dissolvable mint according to the invention.

TABLE 3
Exemplary Mint Formulation Blend

| Component | Ingredient | Amount (wt. %) |
|---|---|---|
| Base | Sorbitol tableting blend | 55.17 |
| | Xylitol tableting blend | 27.58 |
| Lubricant | Magnesium stearate | 0.75 |
| Sweetener | Stevia | 0.4 |
| Flavorant | Peppermint | 0.75 |
| Flavorant | Fona Optify cooling enhancer | 0.1 |
| Bitter blocker | ClearIQ Bitter Blocker | 0.5 |
| Encapsulated CBD | Encapsulated CBD | 14.75 |
| | (10%) on fibersol | |

| | |
|---|---|
| Total | 100.00 |

Preparation Methods

CBD Encapsulation:

a. create oil phase by adding CBD source to medium chain triglyceride oil and heat to maintain dissolution;
b. create aqueous phase consisting of DI water and heat;
c. slowly add oil phase to aqueous phase and mix phases together with a low shear rotor-stator mixer to create a coarse emulsion. Ensure there is no phase separation;
d. pass coarse emulsion through microfluidizer processor a number of times to convert into a nanoemulsion;
e. spray dry nanoemulsion onto maltodextrin;
f. obtain encapsulated CBD powder with 10-20% CBD potency.

Tableting Procedure:

a. Combine all powders (base, sweetener, flavorant, bitter blocker, encapsulated CBD) except lubricant in a non-shearing blender;
b. blend until uniform;
c. add lubricant so that it coats powders;
d. blend long enough to obtain homogeneity
e. press into tablets.

The above tableting procedure results in 1 g mints with a final concentration of 15-20 mg CBD per mint, which was confirmed by 3^{rd} party analytical analysis.

CBD Taste Masking Methods

The present invention also teaches a method of masking the bitter taste of CBD by preparing a powder containing CBD and then combining the powder with a bitter blocker. The powder can be an encapsulated CBD prepared by any of the methods set forth herein. The powder can also be prepared by spray drying a CBD nanoemulsion, as defined herein, onto a substrate to obtain a powder, and then combining the powder with a bitter blocker.

In embodiments of the method, the substrate is maltodextrin. Preferably the maltodextrin a digestion resistant maltodextrin.

In embodiments of the method, the bitter blocker is mushroom mycelia. Preferably, the mushroom mycelia are Cordyceps sinensis mycelia.

1. A solid composition comprising encapsulated cannabidiol (CBD), mushroom mycelia, and flavor.

2. The solid composition of claim 1, wherein the solid composition comprises about 15 wt. % to about 20 wt. % encapsulated CBD.

3. The solid composition of claim 2, wherein the encapsulated CBD contains about 10 wt. % CBD.

4. The solid composition of claim 3, wherein the CBD is selected from the group consisting of: CBD distillate, CBD isolate, full-spectrum CBD oil, and broad-spectrum CBD oil.

5. The solid composition of claim 1, wherein the encapsulated CBD comprises a CBD emulsion spray dried on or in a substrate.

6. The solid composition of claim 5, wherein the substrate is maltodextrin.

7. The solid composition of claim 6, wherein the maltodextrin is digestion resistant.

8. The solid composition of claim 1, wherein the mushroom mycelia are from Cordyceps sinensis mycelia.

9. The solid composition of claim 1, wherein the flavor comprises a sweetener and an optional flavorant.

10. The solid composition of claim 9, wherein the sweetener is stevia.

11. The solid composition of claim 1, wherein solid composition is free of sucrose.

12. The solid composition of claim 1, wherein the solid composition is a tablet.

13. The solid composition of claim 1, wherein the solid composition is orally dissolvable.

14. A method of masking the bitter taste of CBD, comprising:
i) preparing a CBD emulsion comprising a CBD and a carrier oil;
ii) spray drying the emulsion onto a substrate to obtain a powder; and
iii) combining the powder with mushroom mycelia.

15. The method of claim 14, wherein the substrate is maltodextrin.

16. The method of claim 14, wherein the mushroom mycelia are Cordyceps sinensis mycelia.

17. The method of claim 14, wherein the carrier oil is medium chain triglyceride (MCT) oil.

18. A nutraceutical product comprising the solid composition of claim 1.

19. The nutraceutical product of claim 18, wherein the product contains about 1.5% to about 2.0 wt. % CBD.

20. The nutraceutical product of claim 17, wherein the product is a mint.
